(19) United States
(12) Patent Application Publication
(10) Pub. No.: US 2024/0114973 A1
(43) Pub. Date: Apr. 11, 2024

(54) A Method for Controlling the Heating of a Susceptor of an Aerosol-Generating Device Using a Boost Converter

(71) Applicant: JT International SA, Geneva (CH)

(72) Inventors: Branislav Zigmund, Vrbovce (SK); Stefan Lojek, Zilina (SK); Daniel Vanko, Krpelany (SK); Petr Konvicný, Roznov p.R. (CZ); Stanislav Sliva, Ostrava (CZ)

(73) Assignee: JT International SA, Geneva (CH)

(21) Appl. No.: 18/275,534

(22) PCT Filed: Feb. 4, 2022
(86) PCT No.: PCT/EP2022/052755

§ 371 Date: Aug. 2, 2023

(30) Foreign Application Priority Data
Feb. 5, 2021 (EP) 21155429.0

(51) Int. Cl. A24F 40/57; A24F 40/465; H02M 3/156; H02M 7/537; H05B 6/10

(52) U.S. Cl. CPC A24F 40/57; A24F 40/465; H02M 3/156; H02M 7/537; H05B 6/108

(57) ABSTRACT

A method for controlling the heating of a susceptor of an aerosol-generating device is described, the susceptor being inductively heated by an oscillating circuit driven by an inverter, an optional boost converter being connected between a power supply unit and the inverter, the boost converter and being configured to step-up voltage from an input voltage supplied from the power supply unit to an output voltage delivered to the inverter. The method includes a power delivery mode of the aerosol-generating device and a temperature identification mode of the aerosol-generating device in which the amount of power supplied to the inverter is lower than the amount of power supplied during the power delivery mode. The method includes determining the temperature of the susceptor, e.g. based on a determined resonant frequency or resonant capacitor voltage of the oscillating circuit.

### TECHNICAL FIELD

The present disclosure relates generally to a method for controlling the heating of a susceptor of an aerosol-generating device and an aerosol-generating device comprising a controller adapted to implement said method.

### TECHNICAL BACKGROUND

An aerosol-generating device generally comprises at least one reservoir arranged to store an aerosol-generating product. The aerosol-generating product is heated, without burning, in order to generate an aerosol for inhalation.

The aerosol-generating product can be heated using different methods. One method consists in using induction heating. Such an aerosol-generating device thus comprises an induction heating system usually comprising an induction coil, an induction heatable susceptor and a power supply unit.

Thanks to the power supply unit or battery, electrical energy is provided to the induction coil by the inverter. The induction coil thus generates an alternating electromagnetic field. The susceptor couples with the electromagnetic field and generates heat which is transferred, for example by conduction, to the aerosol-generating product. Finally, the heated aerosol-generating product generates an aerosol.

For an optimized operating of the aerosol-generating device, there is a need to seek the highest possible energy efficiency during inductive heating.

In this context, it is known, for example, to use a boost converter for an aerosol-generating device. A boost converter is configured to step-up the voltage supplied by a power supply unit, i.e. to transform a DC voltage into a DC voltage of a higher value. CN209732613U discloses such an aerosol-generating device.

The present disclosure aims at providing an improved method for controlling the inductive heating of a susceptor of an aerosol-generating device and more precisely for improving the energy efficiency using a boost converter.

### SUMMARY OF THE DISCLOSURE

The present disclosure thus relates to a method for controlling the heating of a susceptor of an aerosol-generating device, the susceptor being inductively heated by an oscillating circuit driven by an inverter.

According to a first aspect of the present disclosure, the method comprises a power delivery mode of the aerosol-generating device and a temperature identification mode of the aerosol-generating device in which the amount of power supplied to the inverter is lower than the amount of power supplied during the power delivery mode, wherein the method further comprises determining the temperature of the susceptor based on measurements taken during the temperature identification mode.

A boost converter may be connected between a power supply unit and said inverter, the boost converter being configured to step-up voltage from an input voltage supplied from the power supply unit to an output voltage delivered to the inverter. The power delivery mode may comprise a step of setting the output voltage delivered from the boost converter to the inverter depending on the determined temperature of the susceptor.

An efficient power control is thus possible thanks to a regulation of the voltage delivered to the oscillating circuit depending on the temperature of the susceptor and thus of the temperature of the aerosol-generating product. The determination of the temperature of the aerosol-generating product and the control of the delivered voltage accordingly enable the production of the right amount of aerosol with appropriate substance with a high energy efficiency.

The output voltage can thus vary depending on the desired heating profile which depends itself on other parameters such as the nature or type of the aerosol-generating product.

The boost converter also provides a smooth power control in induction heating which is not easy to control.

The method may comprise a comparison step performed before the setting step, in which the determined temperature of the susceptor is compared with a target temperature, the output voltage being set at a value depending on the determined temperature and the target temperature.

The output voltage can thus vary depending on the target temperature. It is possible for example to control the output voltage such as to reach but not exceed the target temperature. Or on the contrary, it is possible to control the output voltage to overshoot the target temperature.

The aerosol-generating device may comprise a controller configured to control the output voltage of the boost converter in order to bring the temperature of the susceptor to the target temperature, said controller being tuned to be overdamped, and wherein the output voltage is being set at a maximum predefined voltage when the determined temperature of the susceptor is inferior or equal to a threshold value.

The threshold value may range between 60% and 85% of the target temperature.

The aerosol-generating device may comprise a controller configured to control the output voltage of the boost converter in order to bring the temperature of the susceptor to the target temperature, said controller being tuned to be underdamped, and wherein the output voltage is set so that the temperature of the susceptor overshoots the target temperature at the beginning of the heating of the susceptor.

The controller may be a PID controller, a model-based controller and/or a model-predictive controller.

The output voltage may be set substantially equal to or less than a predetermined voltage, e.g. about 8V, when the determined temperature of the susceptor is equal to the target temperature.

The boost converter may be an asynchronous boost converter.

The boost converter may be a synchronous boost converter.

The boost converter may comprise an active switch, said active switch being a MOSFET transistor.

The boost converter may comprise a passive switch, said passive switch being a MOSFET transistor.

The boost converter may be configured to step-up voltage from an input voltage ranging from 3 to 4.2V to a desired output voltage. The desired output voltage will be sufficient to generate appropriate losses in the susceptor for required heating and in some aspects the desired output voltage may be at least equal to 8V. The desired output voltage may depend on the susceptor properties such as resistance, shape and size etc.

If a boost converter is not needed, the inverter may be controlled by a controller to adjust the induction heating during the power delivery mode. For example, the inverter may be periodically enabled and disabled (or periodically controlled to be in an On-state and an Off-state) with a duty cycle that can be varied to control the heating of the susceptor. Such operation can be referred to as a “global” pulse width modulation (PWM) control scheme where the time for which the inverter is enabled (or “pulse width”) is varied. The inverter may comprise two transistors. During the periods when the inverter is enabled (or in an On-state) the transistors can be operated at a predetermined duty cycle. Both transistors are turned off during the periods when the inverter is disabled (or in an Off-state).

The inverter may comprise two transistors. Both transistors are preferably operated during power delivery mode.

The oscillating circuit may comprise a coil circuit and a susceptor circuit. The coil circuit may be an LLC circuit or an LC circuit, for example, and will normally include at least one inductor or coil and at least one capacitor. In some aspects of the present disclosure, an LC circuit may be preferred because it includes fewer power dissipative components. In the LLC circuit, the additional filter inductor may increase resistive power losses and increase the required battery voltage. It can also lead to higher switching losses in the inverter.

The determination of the temperature of the susceptor may be based on a determined resonant frequency of the oscillating circuit.

Said step of temperature determination may comprise the following sub-steps:
operating only one of the two transistors of the inverter during the temperature identification mode;
determining the resonant frequency of the oscillating circuit during the temperature identification mode; and
determining the temperature of the susceptor based on said determined resonant frequency.

The determination of the temperature of the susceptor may be based on a determined maximum value of an indicative electrical value in the oscillating circuit, e.g. the voltage across a capacitor of the coil circuit.

Said step of temperature determination may comprise the following sub-steps:
determining the maximum value of an indicative electrical value in the oscillating circuit for a range of frequencies during the temperature identification mode, e.g. while sweeping the frequencies in a range between a minimum frequency fₘᵢₙ and a maximum frequency fₘₐₓ;
determining a global maximum value from the determined maximum values; and
determining the temperature of the susceptor based on said global maximum value.

Said step of temperature determination may further comprise the sub-step of operating both of the two transistors of the inverter during the temperature identification mode at a reduced duty cycle, e.g. about 10% to 15%. More particularly, the duty cycle of the inverter during the temperature identification mode is preferably lower than the duty cycle of the inverter during the power delivery mode. The duty cycle during the temperature identification mode is preferably reduced to a minimum value to have as little power delivered to the susceptor as possible. For example, the susceptor heat-up during one frequency sweep may be kept below 1° C., which is enough to ensure high performance.

Said sub-steps of determining the global maximum value and/or the temperature of the susceptor based on said global maximum value need not be carried out during the temperature identification mode. In other words, the measurements needed to determine the susceptor temperature are taken during the temperature identification mode, but the processing for the actual determination of the temperature of the susceptor may take place after the temperature identification mode has ended.

The power delivery mode and the temperature identification mode may be alternated during operating of the aerosol-generating device.

The temperature identification mode may be run at regular intervals of time.

According to a second aspect of the present disclosure there is provided a method for controlling the heating of a susceptor of an aerosol-generating device, the susceptor being inductively heated by an oscillating circuit driven by an inverter, a boost converter being connected between a power supply unit and said inverter, the boost converter being configured to step-up voltage from an input voltage supplied from the power supply unit to an output voltage delivered to the inverter, wherein said method comprises a power delivery mode of the aerosol-generating device and a temperature identification mode of the aerosol-generating device in which the amount of power supplied to the inverter is lower than the amount of power supplied during the power delivery mode, the method further comprising a step of determining the temperature of the susceptor, and setting the output voltage delivered from the boost converter to the inverter during the power delivery mode depending on the determined temperature of the susceptor.

Other features of the aerosol-generating device and the method are as described above.

According to a third aspect of the present disclosure, there is provided an aerosol-generating device comprising:
a power supply unit;
an induction heatable susceptor;
an oscillating circuit arranged to generate a time varying electromagnetic field for inductively heating the susceptor;
an inverter configured to drive the oscillating circuit;
an optional boost converter connected on the one side to the power supply unit and on the other side to the inverter; and
a controller adapted to implement the method for controlling the heating of the susceptor as previously described.

### BRIEF DESCRIPTION OF THE DRAWINGS

Other particularities and advantages of the present disclosure will also emerge from the following description.

In the accompanying drawings, given by way of non-limiting examples:

FIGS. 1a, 1b represent schematically part of an aerosol-generating device 1 according to two embodiments of the present disclosure;

FIG. 2 represents schematically the electronic circuitry of the aerosol-generating device;

FIG. 3 represents schematically a control loop system according to an embodiment of the present disclosure;

FIG. 4a represents an oscillating circuit and inverter of the FIG. 2;

FIG. 4b represents an oscillating circuit and inverter according to another embodiment of the present disclosure;

FIG. 5a represents separately a boost converter circuit of the FIG. 2;

FIG. 5b represents the boost converter circuit according to another embodiment of the present disclosure;

FIG. 6 represents a linear dependency between the resonant frequency of an oscillating circuit and the temperature of a susceptor of the aerosol-generating device;

FIG. 7 represents schematically a method for controlling the heating by induction of the susceptor of the aerosol-generation device;

FIG. 8 represents an example of temperature control that can be implemented in the aerosol-generating device; and

FIG. 9 represents a dependency between a voltage of an oscillating circuit and the temperature of a susceptor of the aerosol-generating device.

### DETAILED DESCRIPTION OF EMBODIMENTS

Embodiments of the present disclosure will now be described by way of example only and with reference to the accompanying drawings.

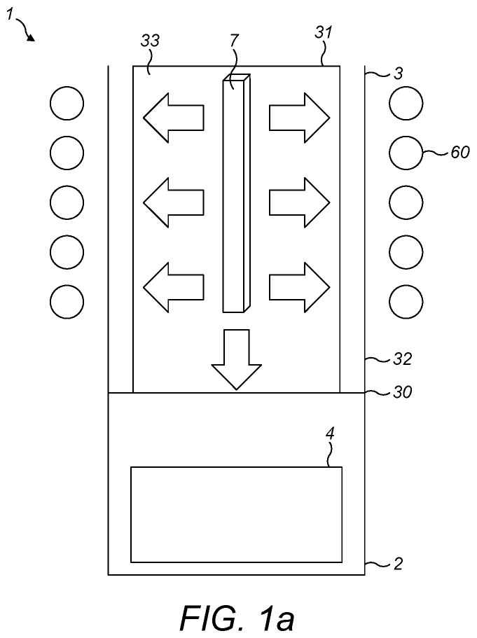

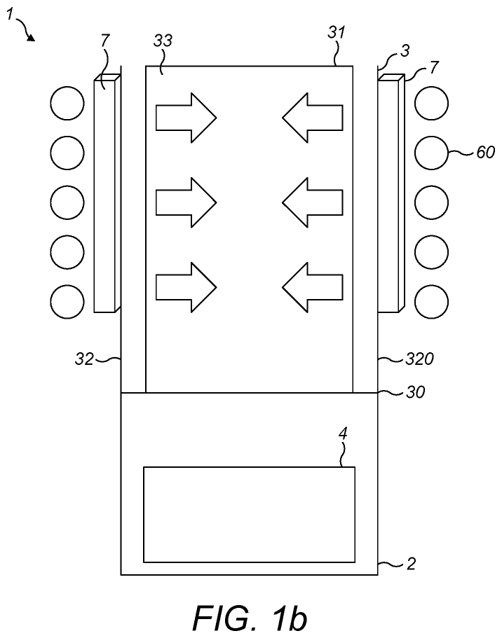

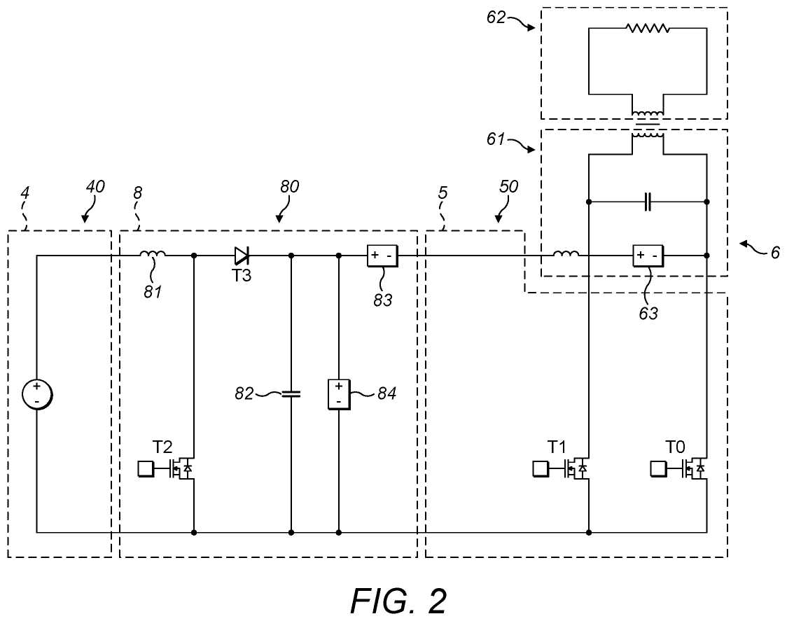

FIGS. 1a and 1b represent schematically part of an aerosol-generating device 1 according to two different embodiments of the present disclosure. Both FIGS. 1a, 1b show schematically the mechanical configuration of the aerosol-generating device 1, whereas FIG. 2 represents an example of the electronic circuitry of the aerosol-generating device 1.

The aerosol-generating device 1 comprises a main body 2 and a cartridge 3.

The cartridge 3 comprises a first end 30 configured to engage with the body 2 and a second end 31 arranged as a mouthpiece portion (not shown) having a vapor outlet.

The cartridge 3 further comprises at least one reservoir 32 arranged to store an aerosol-generating product 33. The cartridge 3 may be disposable.

The reservoir 32 is arranged to receive a correspondingly shaped aerosol-generating product 33. The aerosol-generating product 33 and/or the reservoir may be a disposable article or stick.

The term aerosol-generating product is used to designate any material that is vaporizable in air to form an aerosol. Vaporization is generally obtained by a temperature increase up to the boiling point of the vaporization material, such as at a temperature up to 400° C., preferably up to 350° C. The vaporizable material may, for example, be in liquid form, solid form, or in a semi liquid form, thus comprise or consist of a liquid, tobacco, gel, or wax or the like, or any combination of these.

The mouthpiece is removably mounted to allow access to the reservoir for the purposes of inserting or removing the aerosol-generating product 33.

The aerosol-generating device 1 comprises an induction heating system configured to enable the heating of the aerosol-generating product 33.

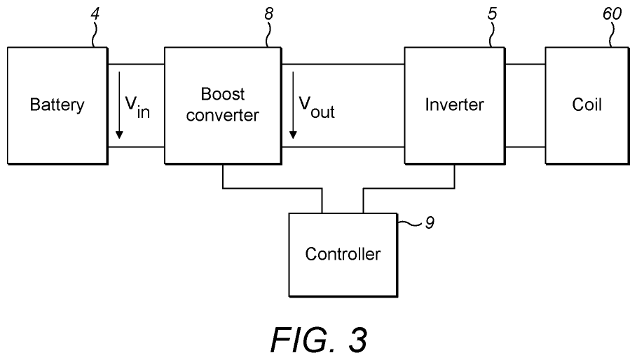

The induction heating system comprises a power supply unit or battery 4 as well as an inverter 5 and a controller 9 (visible on FIG. 3), generally disposed in the body 2.

The controller 9 is configured to operate other electronic components among which is the inverter 5.

The inverter 5 is arranged to convert a direct current from the battery 4 into an alternating high-frequency current. The inverter 5 comprises here two switches or transistors, T0, T1. The transistors T0, T1 are operated at the same frequency and at a predetermined duty cycle. In particular, the duty cycle of the two transistors T0, T1 of the inverter 5 is equal to 50%. A duty cycle of 50% during the power delivery mode is generally preferred so that the transistors T0, T1 have symmetrical loading, but it will be understood that other duty cycles are possible. It is also possible to operate the transistors T0, T1 with a variable duty cycle, e.g. a duty cycle of about 20% to 80%. This may be appropriate if there is no boost converter to adjust the voltage supplied to the inverter and there is no other way of controlling power delivery.

The induction heating system further comprises an oscillating circuit 6. The oscillating circuit comprises an inductance provided by a coil 60.

The coil 60 is here a helical induction coil which extends around the reservoir 32. The induction coil 60 is energized by the battery 4 and the controller 9. The controller 9 is configured to control the operating frequency fₒₚ at which the oscillating circuit 6 is driven.

The induction heating system also comprises one or more induction heatable susceptors 7. A susceptor is an element made in an electrically conducting material and used to heat a non-electrically conducting material or product.

The induction heatable susceptor 7 can be in direct or indirect contact with the aerosol-generating product 33, such that when the susceptor 7 is inductively heated by the induction coil 60, heat is transferred from the susceptor 7 to the aerosol-generating product, to heat the aerosol-generating product and thereby produce an aerosol.

In the represented example shown in FIG. 1a, the susceptor 7 extends within the reservoir 32 with the aerosol-generating product 33. The susceptor 7 is preferably arranged inside the aerosol generating product 33.

In another embodiment shown in FIG. 1b, the susceptor 7 extends outside the aerosol generating product 33. The susceptor 7 preferably extends along lateral walls 320 of the reservoir 32.

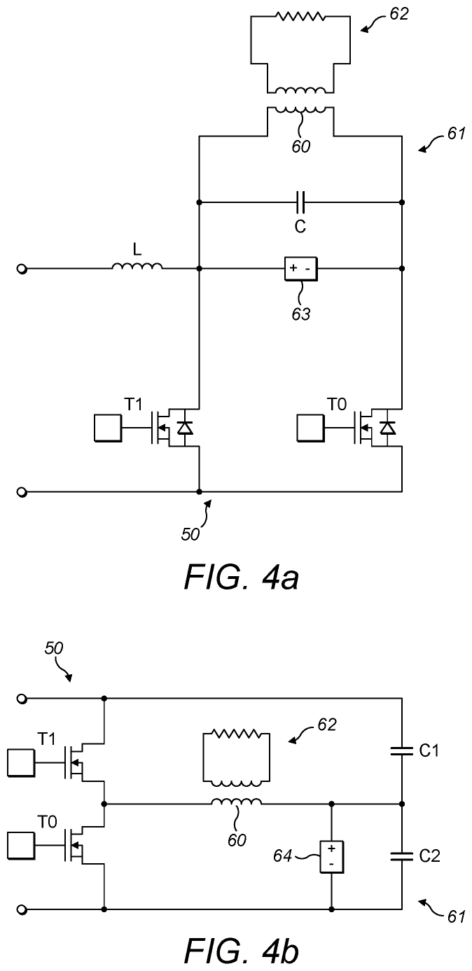

FIG. 2 represents the battery circuit 40, the inverter circuit 50, the oscillating circuit 6 comprising a coil circuit 61 and a susceptor circuit 62. The oscillating circuit 6 is represented separately on FIG. 4a. FIG. 4b represents another example of an oscillating circuit that is also appropriate.

In the embodiment of FIG. 4a, the coil circuit 61 is an LCC circuit with an additional inductor. A voltage sensor 63 is adapted to measure the voltage across the capacitor C of the coil circuit 61.

In the embodiment of FIG. 4b, the coil circuit 61 is an LC circuit. A voltage sensor 64 is adapted to measure the voltage across one of the capacitors C2 of the coil circuit 61.

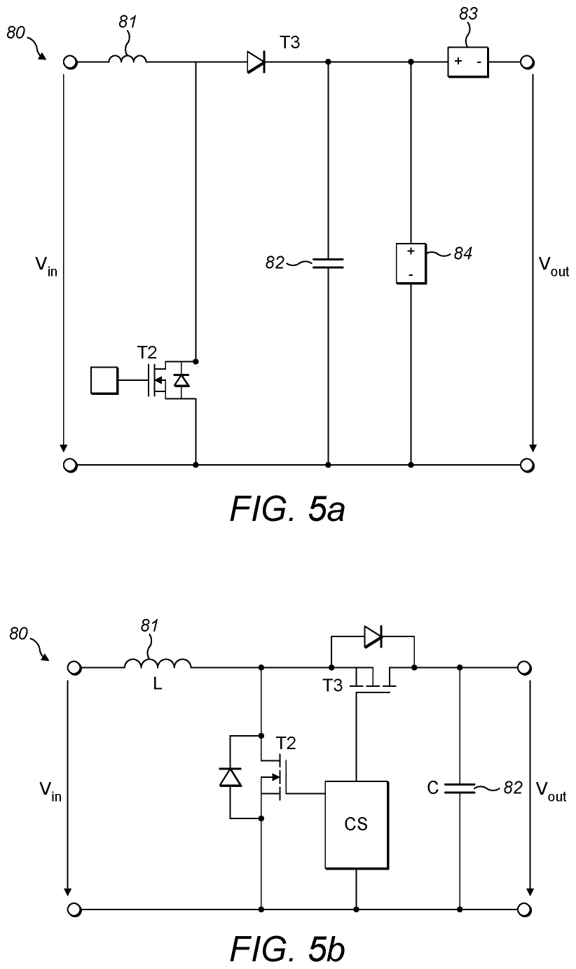

The aerosol-generating device 1 also comprises a boost converter 8, of which an example of a circuit 80 is represented at FIG. 2. In particular, FIG. 2 includes an example of a boost converter 8 that can be used in the aerosol-generating device, and that is represented separately on FIG. 5a. FIG. 5b represents another example of a boost converter 8 that is also appropriate.

The boost converter 8 is on the one part connected to the battery 4 and to the other part connected to the inverter 5. In some aspects of the present disclosure, the boost converter 8 can be omitted and the inverter 5 is connected directly to the battery 4 or other power source. If there is no boost converter, during a power delivery mode described below, the induction heating of the susceptor 7 may be controlled by a controller using a “global” PWM control scheme. The inverter 5 may operated in an On-state where the transistors T0, T1 are switched on an off at a predetermined duty cycle. In particular, the duty cycle of the two transistors T0, T1 of the inverter 5 is equal to 50%. When the inverter 5 is operated in an Off-state, the two transistors T0, T1 are switched off. The overall duty cycle of the “global” PWM control scheme can be controlled to vary the heating of the susceptor 7 during the power delivery mode.

The boost converter 8 is configured to step-up the voltage, i.e. to transform a DC voltage into a DC voltage of a higher value. More precisely, the boost converter 8 is configured to step-up voltage from an input voltage Vᵢₙ supplied from the power supply unit 4 to a higher output voltage Vₒᵤₜ delivered to the inverter 5.

The boost converter 8 is an advantageous solution for increasing voltage with minimal space.

A boost converter is a type of switch mode power supply. In particular, it uses a main switch, for example a transistor to turn part of the circuit on and off at a certain speed.

The boost converter 8 comprises an active switch T2 and a passive switch T3.

The active switch T2, or main switch, is in both represented examples a MOSFET transistor (Metal-oxide Semiconductor Field Effect Transistor).

In the embodiment of FIG. 5a, the passive switch T3, or auxiliary switch, is a diode. The boost converter is thus an asynchronous boost converter.

In the embodiment of FIG. 5b, the passive switch T3 is a MOSFET transistor. The boost converter 8 is thus a synchronous boost converter.

The boost converter 8 further comprises an inductor 81 and a capacitor 82.

The boost converter circuit 80 further comprises here two sensors, a current sensor 83 and a voltage sensor 84. The current sensor 83 is adapted to measure the output current delivered by the boost converter 8. The voltage sensor 84 is adapted to measure the output voltage Vₒᵤₜ delivered by the boost converter 8.

The principle of the boost converter consists of two distinct states, namely an On-state and an Off-state. In the On-state, the main switch T2 is on, and the inductor 81 is charged. In the Off-state, the main switch T2 is off and the energy of the inductor 81 starts to dissipate.

The boost converter 8 is also characterized by a duty cycle D. The duty cycle D represents the fraction of the commutation period T during which the main switch T2 is On. Therefore, D ranges between 0 and 1.

The average output voltage Vₒᵤₜ is directly related to the input voltage Vᵢₙ and the duty cycle D as shown by the following relationship: Vₒᵤₜ=Vᵢₙ*1/1−D.

The boost converter is configured here to step-up voltage from an input voltage Vᵢₙ ranging from 3 to 4.2V to a higher output voltage Vₒᵤₜ. The output voltage Vₒᵤₜ is preferably at least equal to 8V.

The controller 9 is configured here to control the boost converter 8, in particular to control the output voltage delivered to the inverter 5.

FIG. 3 shows an example of a control loop system that can be used in the present disclosure. The controller 9 is connected on the one side to the inverter 5 and on the other side to the boost converter 8.

The controller 9 is for example a proportional-integral-derivative controller (PID controller).

For a more advanced control and better performance, other topologies or controller types can be used. The controller 9 can be for instance a model-based controller. Such a controller has the advantage of taking into account the dynamic response of the system which changes with operating conditions. The model-based controller yields significantly better performance and exhibits a much lower sensitivity to variation in system properties compared to a regular PID controller. It enables for instance a rapid ramping up or ramping down of the temperature when needed.

In yet another particular embodiment, the controller 9 can be a model-predictive controller or a model-based predictive controller. Such a controller is also able to represent the behaviour of a dynamic system and further uses a model of the system to make predictions about the system's future behaviour.

A hybrid or mixed control may also be used. For example, if the aerosol-generating device includes a boost converter 8, the boost converter may be controlled by the controller 9 for some operations of the aerosol-generating device (e.g., during pre-heating) while for other operations (e.g., during a vaping phase) the boost converter may be bypassed or disabled and the induction heating of the susceptor 7 is controlled by the inductor, e.g. using the “global” PWM control scheme mentioned above. During pre-heating, more power is needed and the boost converter 8 would be beneficial in providing a higher output voltage for the inverter 5. A higher voltage means a lower current is needed to achieve the same power, which can reduce losses. Afterwards, during a vaping phase, less power is needed and there is no need for the boost converter 8. Conducting losses can therefore be reduced by bypassing the boost converter 8.

The method for controlling heating of the susceptor 7 of the aerosol-generating device 1 first comprises a step of determination of the temperature of the susceptor 7.

The temperature of the susceptor 7 can be determined using any appropriate method. For example, the temperature of the susceptor 7 can be determined by first determining the resonant frequency of the oscillating circuit 6.

Indeed, the resonant frequency fᵣ of the oscillating circuit is influenced by the values of inductance L, resistance R and capacitance C, and for the LLC circuit shown in FIG. 4a is given as follows:

$${f}_{r} = \frac{1}{2 π} \sqrt{\frac{1}{L C} - {( \frac{R}{L} )}^{2}}$$

Furthermore, the resonant frequency fᵣ of the oscillating circuit 6 depends:
on the exact positioning of the susceptor 7 with respect to the inductance coil 60 of the oscillating circuit 6; and
on the resistance of the susceptor 7 which varies with the temperature of the susceptor.

The variation of the resistance can also be influenced by the manufacturing tolerance.

Therefore, the resonant frequency fᵣ can be used to track the change in total resistance and thus the temperature of the susceptor 7.

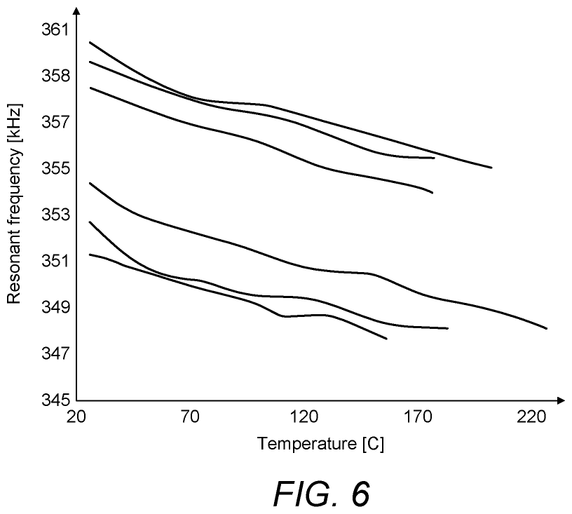

More specifically, the resonant frequency fᵣ varies linearly with the temperature as shown in FIG. 6. A functional form describing the temperature T of the susceptor 7 as a function of frequency characteristic F can be written as F(T)=aT+b, where ‘a’ and ‘b’ are constant parameters of the functional form. The parameter ‘a’ corresponds to the slope value of the curve of frequency. The parameter ‘b’ corresponds to the y-intercept.

The different curves of FIG. 6 represent the variation of the frequency of the oscillating circuit in function of the temperature and of the position of the susceptor 7. Indeed, as explained above, the resonant frequency depends on the position of the susceptor 7 with respect to the oscillating circuit. This therefore modifies the y-intercept of the curve of the frequency. This appears clearly in FIG. 6 where the slope ‘a’ is the same for all the curves and the y-intercept is different from one curve to another.

In the illustrated embodiment, the y-intercept or b parameter corresponds to an initial resonant frequency fᵢ of the resonant circuit. The initial resonant frequency fᵢ shall refer to the resonant frequency of the oscillating circuit before heating of the susceptor 7. In other words, it corresponds to resonant frequency when the susceptor 7 is at ambient temperature, i.e. around 20° C.

The illustrated curves thus show that it is possible to take into account an improper insertion of the susceptor 7 in the aerosol-generating device.

The temperature of the susceptor 7 can be determined by first determining a resonant capacitor voltage of the oscillating circuit 6. Indeed, the resonant capacitor voltage V_{c} of the oscillating circuit at resonant frequency is influenced by the values of inductance L, resistance R and supply voltage Vₛ, and for the LC circuit shown in FIG. 4b is given as follows:

$${V}_{c} = \sqrt{( \frac{L}{C} )} * ( \frac{{V}_{s}}{R} )$$

The resonant capacitor voltage therefore depends on the resistance of the susceptor 7 which varies with the temperature of the susceptor.

Preferably, with the boost converter 8, the determination step is performed at a low power supply, i.e. with a low output voltage Vₒᵤₜ. Low output voltage Vₒᵤₜ shall mean less than or equal to 8V. Preferably, the output voltage at low output supply is substantially equal to 8V. Performing the determination step at a low power supply enables a more accurate determination of the susceptor's temperature. Moreover, it enables minimum energy consumption, which is advantageous since energy conversion for heating is not the purpose of this step.

The method for controlling the heating of the susceptor 7 further comprises a comparison step performed in which the determined temperature T_{d} of the susceptor 7 is compared with a predefined or target temperature Tₜ. A target temperature shall mean a predefined and preset temperature that is aimed for and that should be maintained for correct aerosolisation of the aerosol-generation product.

It shall be understood that the controller or the aerosol-generating device 1 can be configured to store the predefined or target temperature Tₜ of the susceptor 7. The controller or the aerosol-generating device can also comprise a comparator that compares the determined temperature T_{d} to the stored target temperature Tₜ.

Then the method comprises a step of setting of the output voltage Vₒᵤₜ delivered from the boost converter 8 to the inverter 5 depending on the determined temperature T_{d} of the susceptor 7.

The output voltage Vₒᵤₜ of the boost converter 8 can be adjusted for the temperature of the susceptor 7 to reach and then be maintained at the target temperature Tₜ.

The controller operates in a closed-loop adjusting the output voltage Vₒᵤₜ depending on the determined temperature T_{d} of the susceptor 7.

For example, as long as the determined temperature T_{d} is below the target temperature Tₜ, a high power supply to the inverter 5 is maintained. High power supply shall mean a high output voltage Vₒᵤₜ. A high output voltage Vₒᵤₜ is higher than 8V. When the target temperature Tₜ is approached, the power supply can be reduced. Once the target temperature Tₜ is reached, the power supply is set very low. In other words, the output voltage Vₒᵤₜ is set at a low value, preferably less than or equal to 8V.

The heating process and accordingly the control of the output voltage depend on design choices and changes depending on parameters such as the nature or type of the aerosol-generating product, the desired heating profile, etc.

Therefore, by repeating the determination step and the setting step during operation of the aerosol-generating device, the voltage delivered to the oscillating circuit 6 can be adjusted frequently. This enables a good power control and energy efficiency.

For example, the steps of determination and setting are repeated at certain intervals during operation of the aerosol-generating device. The steps of determination and setting are repeated at regular intervals.

In another embodiment, the steps of determination and setting can be continuously repeated during operation of the aerosol-generating device.

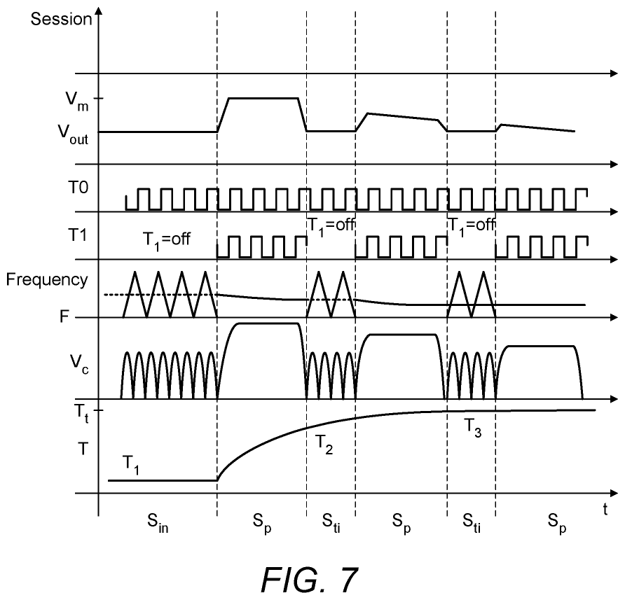

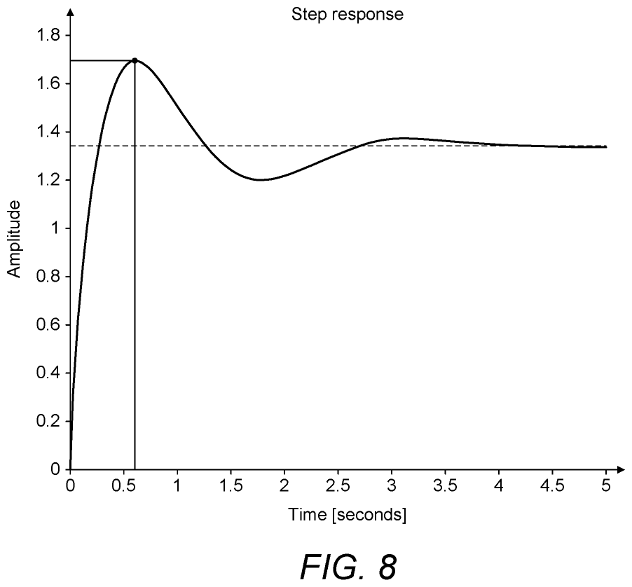

An example of implementation of such a method for controlling the heating of the susceptor 7 is represented at FIG. 7.

On this figure, T refers to the temperature of the susceptor 7, V_{c} the voltage across the capacitor of the oscillating circuit, T0, T1 are the two transistors of the inverter 5, the frequency F of the oscillating circuit 6, and Vₒᵤₜ is the output voltage delivered by the boost converter 8. All these parameters are represented as a function of time.

First, an initial resonant frequency fᵢ of the oscillating circuit is determined. This first step is referenced as Sᵢₙ in FIG. 7 and also referred to as an initialization step. The initialization step Sᵢₙ is performed when the susceptor 7 is at ambient temperature, i.e. before heating it.

For determining the initial resonant frequency fᵢ, a low power energy is supplied to the oscillating circuit 6. In particular, only the transistor T0 of the inverter 5 operates, the transistor T1 being off. The output voltage Vₒᵤₜ of the boost converter is set at a low value, preferably equal to or less than 8V. More preferably, the output voltage Vₒᵤₜ is equal to 8V.

The operating frequency fₒₚ of the inverter 5 is then set at the determined initial resonant frequency fᵢ. The method for controlling the heating of the susceptor 7 further comprises a power delivery mode Sₚ and a temperature identification mode S_{Ti}.

The power delivery mode Sₚ is performed during heating of the susceptor 7. During this mode, both transistors T0, T1 of the inverter 5 are operated. The output voltage Vₒᵤₜ is normally set at a high value. Namely, the output voltage Vₒᵤₜ is set at a value greater than 8V.

While heating the susceptor 7, the resonant frequency fᵣ is continuously tracked. Indeed, resonant frequency changes during the functioning of the aerosol-generating device. Furthermore, operation at the resonant frequency fᵣ ensures the highest possible energy efficiency.

Therefore, the controller tracks the resonant frequency fᵣ and adjusts the actual operating frequency fₒₚ during heating accordingly in power delivery mode Sₚ. In other words, the operating frequency fₒₚ is thus continuously updated in order to correspond to the resonant frequency of the oscillating circuit.

Since the resonant frequency is continuously tracked, the temperature can be continuously determined using the curves of FIG. 6.

Using this method for controlling the heating, the temperature of the susceptor 7 can be continuously monitored during power delivery mode Sₚ.

However, a better and more accurate determination of the temperature of the susceptor 7 is needed. This is enabled by the temperature identification mode S_{Ti} performed at certain intervals of time.

For doing so, after interrupting power supply, a low power energy is supplied to the oscillating circuit. In particular, only the transistor T0 operates and the transistor T1 is cut off. Moreover, the output voltage Vₒᵤₜ is lowered. The output voltage Vₒᵤₜ is lowered to a value equal to or less than 8V.

Then, the resonant frequency is determined. The temperature of susceptor 7 can then be determined using the curves represented at FIG. 6. In practice, the same corresponding curve of the initial resonant frequency is used for determining the temperature of the susceptor. In this disclosure, as explained above, the resonant frequency fᵣ varies linearly with the temperature as shown in FIG. 6. A functional form describing the temperature T of the susceptor 7 as a function of frequency characteristic F can be written as F(T)=aT+b, where ‘a’ and ‘b’ are constant parameters of the functional form. The parameter ‘a’ corresponds to the slope value of the curve of frequency. The parameter ‘b’ corresponds to the y-intercept. The frequency range is from about 300 kHz to about 700 kHz, but may also be from about 100 kHz to about 700 kHz.

The curves as represented on FIG. 6 are adapted after initial resonant frequency determination. The curves can be then shifted or not depending on the equation implemented in the controller.

In another embodiment the curves can be implemented as a look-up table. The look-up table can be registered in the memory of the aerosol-generating device.

Thus, at certain intervals, thanks to the temperature identification mode S_{Ti}, the temperature of the susceptor is accurately identified in order to maintain it at the target temperature Tₜ.

Reducing power delivered to the oscillating circuit 6 during temperature identification mode S_{Ti} enables avoiding power delivery to the susceptor 7. In this manner, the influence on the temperature of the susceptor 7 is reduced, which enables a better estimate of the temperature of the aerosol-generating product 33.

The power delivery mode Sₚ and the temperature identification mode S_{Ti} are alternated.

The temperature identification mode S_{Ti} can be repeated at regular intervals for accurate temperature determination.

In the represented example, the power delivery mode Sₚ and the temperature identification S_{Ti} mode are regularly repeated and alternated. But the duration of the power delivery mode Sₚ and the temperature identification mode S_{Ti} may vary during functioning of the aerosol-generating device. It may be beneficial to reduce how often the temperature identification modes S_{Ti} are carried out depending on the operating factors. For example, it may be beneficial to extend the length of the power delivery mode Sₚ during early phases of heat-up and this would reduce the frequency at which the temperature identification modes S_{Ti} are carried out.

The duration of each power delivery mode Sₚ may be in the range of about 30 to 200 ms, for example.

The duration of each temperature identification mode S_{Ti} can be very short, e.g. in the range of about 2 to 20 ms, in order to get a stable temperature determination of the susceptor. The duration of each temperature identification mode S_{Ti} may depend on other operating factors such as the frequency range that needs to be swept and the required resolution. In some cases, the duration of a particular temperature identification mode can be longer than about 20 ms, for example as long as about 120 ms for a frequency sweep over the broader frequency range mentioned below at high resolution. The first temperature identification mode may be longer than the subsequent temperature identification modes to allow for a broader frequency range (e.g. from 100 kHz to 700 kHz) with subsequent temperature identification modes using a narrower frequency range (e.g., from 350 kHz to 450 kHz). An initial frequency sweep may be carried out over a wide frequency range at low resolution to identify an approximate resonant frequency, with a subsequent frequency sweep being carried out over a narrower frequency range at high resolution for more precise temperature estimation. The narrower frequency range can be targeted on the approximate resonant frequency identified in the initial frequency sweep.

The variation in the temperature of the susceptor during each temperature identification mode may be less than about 1° C.

FIG. 7 shows the susceptor 7 temperature increasing with time thanks to induction heating. The temperature of the susceptor 7 increases until reaching the predefined or target temperature Tₜ.

Temperature of the susceptor 7 is here controlled using a smooth (slow or overdamped) control. In other words, the controller 9 is tuned to be overdamped. Overdamped shall mean that the damping ratio is strictly greater than 1. The temperature of the susceptor 7 thus increases slowly until the target temperature Tₜ. Tuning the controller 9 to be overdamped, prevents or at least reduces overshoot of temperature.

The controller applies therefore the adequate output voltage Vₒᵤₜ to bring the temperature of the susceptor at the desired temperature.

For example, as long as the determined temperature is below the target temperature, the power supply to the inverter 5 is maintained at high output voltage Vₒᵤₜ. When the target temperature Tₜ is approached, the power supply can be reduced. For example, a threshold is preset which when being exceeded, the temperature is considered to approach the target temperature Tₜ. Once the target temperature is reached, power supply is set very low. Namely, the output voltage Vₒᵤₜ is set at a low value.

When using the overdamped temperature control as in FIG. 7, as long as the determined temperature is inferior to a preset percentage of the target temperature, the output voltage supplied to the inverter 5 can be stepped-up to a maximum predefined voltage Vₘ. Preferably, the threshold or preset percentage is between 60% and 85% of the target temperature Tₜ. The threshold depends on other parameters, in particular on the speed of the heating-up of the susceptor. For example, if a pre-heating or a first-puff time is set to be very fast, e.g. of two seconds, the lower limit, namely around 60% of the target temperature is preferred. Indeed, this avoids having an overshoot due to thermal lag of the temperature determination. Pre-heating or first puff shall mean the first period of time in each use of the aerosol-generating device, namely when the user has his first puff.

In the first represented power delivery mode Sₚ at FIG. 7, the voltage is boosted until reaching a voltage value corresponding to the maximum predefined voltage Vₘ while the temperature increases but remains inferior to the target temperature Tₜ.

When the target temperature Tₜ is approached, the output voltage is reduced. In particular, the output voltage is set at a lower voltage than the maximum predefined voltage Vₘ when the determined temperature of the susceptor is superior to the threshold or preset percentage of the target temperature. Thus, in the second power delivery mode Sₚ at FIG. 7, the boost voltage or output voltage is reduced as the temperature of the susceptor 7 approaches the target temperature Tₜ.

Once the target temperature is reached, power supply is set very low. Thus in the third power delivery mode Sₚ of FIG. 7, the output voltage value is once again reduced. Preferably the output voltage is reduced to be equal to or less than 8V.

Of course, this power control is only provided as a way of an example. In another embodiment, the output voltage Vₒᵤₜ can be maintained at the maximum predefined voltage Vₘ even though the target temperature is reached, provoking therefore an overshoot of the temperature of the susceptor. On the contrary, in a safe mode, the output voltage Vₒᵤₜ can be always maintained lower than the maximum predefined voltage Vₘ.

Furthermore, other ways of controlling temperature, i.e. different from an overdamped control, can also be used. For example, temperature of the susceptor 7 can be controlled using a fast underdamped control, as the one represented on FIG. 8. In other words, the controller 9 is tuned to be underdamped. Underdamped shall mean that the damping ratio is strictly less than 1. The controller 9 thus overshoots slightly to reach the target temperature Tₜ more quickly.

In the example of FIG. 8, the controller 9 is configured to overshoot the temperature of the susceptor 7 for a short period of time at the beginning of use of the aerosol-generating device, namely at the pre-heating. In the represented example, the overshoot is reached at around 0.6 seconds.

Tuning the controller 9 to be underdamped, improves the first puff. In such a fast control, the first puff is to be improved while ensuring that some physical limitations are not broken. The physical limitations can be for example no tobacco combustion or no deterioration of the materials of the aerosol-generating device or their assembly.

The determination of the temperature of the susceptor 7 may be based on a determined maximum value of the voltage across a capacitor of the coil circuit 61. For the LLC circuit shown in FIG. 4a, for example, the voltage across the capacitor C may be measured by the voltage sensor 63 during each temperature identification mode S_{Ti}. Similarly, for the LC circuit shown in FIG. 4b, for example, the voltage across the capacitor C2 may be measured by the voltage sensor 64 during each temperature identification mode S_{Ti}.

During each temperature identification mode S_{Ti}, resonant peak capacitor voltage detection is carried out. The inverter 5 is controlled to sweep the frequencies in a range between a minimum frequency fₘᵢₙ and a maximum frequency fₘₐₓ while the voltage across the capacitor is measured. The minimum frequency fₘᵢₙ may be about 350 kHz and the maximum frequency fₘₐₓ may be about 450 kHz, for example. The frequency sweep may be carried out at a particular resolution, where a higher resolution means that voltage measurements are taken for more detection frequencies within the specific frequency range and vice versa. The voltage measurements from the voltage sensor 63 or 64 may be processed or conditioned before the peak voltage for each frequency is detected, e.g. the voltage measurements may be multiplied by a gain and/or processed to remove any DC offset so that only the AC signal is considered. The peak capacitor voltage for each frequency is then detected using a high speed peak detector. For example, V_{c1} is the maximum detected positive capacitor voltage at frequency f₁, V_{c2} is the maximum detected positive capacitor voltage at frequency f₂, V_{c3} is the maximum detected positive capacitor voltage at frequency f₃, and so on for all of the detection frequencies between fₘᵢₙ and fₘₐₓ. This peak detection processing can be considered as an extraction of the voltage envelope. The global peak capacitor voltage is then selected or picked from the determined peak capacitor voltages V_{c1}, V_{c2}, V_{c3}, . . . , V_{cn} as the resonant capacitor voltage using a known pick detection function. The global peak capacitor voltage is the highest of the peak capacitor voltages detected across all of the frequencies swept during the particular temperature identification mode S_{Ti}.

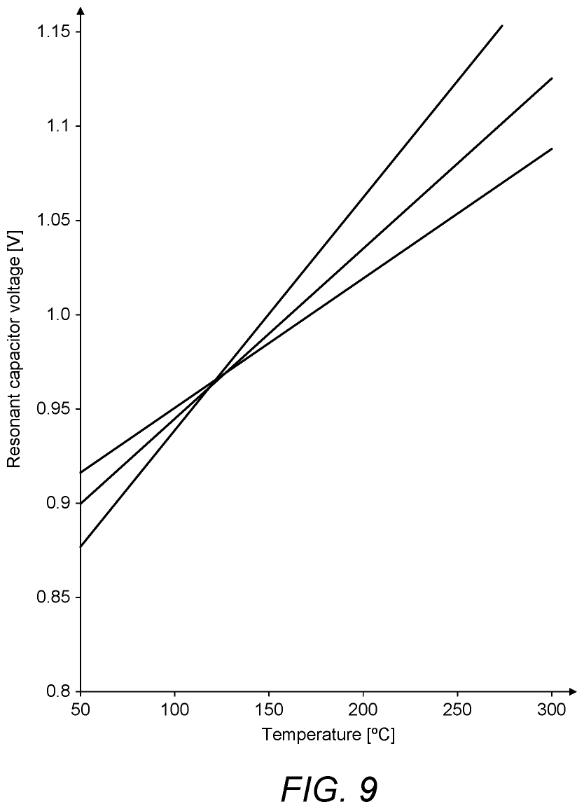

The global capacitor peak voltage (or resonant capacitor voltage) can then be used to determine the temperature of the susceptor 7. More specifically, the resonant capacitor voltage varies with the temperature as shown in FIG. 9. A functional form describing the temperature of the susceptor 7 as a function of the capacitor voltage characteristic can be determined. A functional form can also be determined that describes the temperature of the susceptor 7 as a function of the frequency at which the resonant capacitor voltage is obtained, i.e. the particular frequency at which the highest peak capacitor voltage is measured during the frequency sweep.

During each temperature identification mode S_{Ti}, the transistors T0, T1 operate at a reduced duty cycle, e.g. about 10% to 15% so as to minimise the heating of the susceptor 7.

The determined temperature of the susceptor 7 can be used to adjust the induction heating during a subsequent power delivery mode Sₚ.

It should be understood that various changes and modifications to the presently preferred embodiments described herein will be apparent to those skilled in the art. Such changes and modifications can be made without departing from the spirit and scope of the present disclosure and without diminishing its attendant advantages. It is therefore intended that such changes and modifications be covered by the appended claims.

For example, it will be appreciated that other functional forms may be used for the dependency between the resonant frequency and the temperature of the susceptor. For example non-linear functional forms such as polynomial functions parameterized as appropriate can be used.

The present disclosure thus provides a method for controlling inductive heating in an aerosol-generating device that enables optimizing energy efficiency. Furthermore, the output voltage delivered to the oscillating circuit can be regulated in order to obtain desired temperature profile of the aerosol-generating product.

Any combination of the above-described features in all possible variations thereof is encompassed by the present disclosure unless otherwise indicated herein or otherwise clearly contradicted by context.

Unless the context clearly requires otherwise, throughout the description and the claims, the words “comprise”, “comprising”, and the like, are to be construed in an inclusive as opposed to an exclusive or exhaustive sense; that is to say, in the sense of “including, but not limited to”.

References used for the figures

| | |
|---|---|
| 1 | Aerosol-generating device |
| 2 | Main body |
| 3 | Cartridge |
| 30 | First end of the cartridge |
| 31 | Second end of the cartridge |
| 32 | Reservoir |
| 33 | Aerosol-generating product |
| 4 | Battery |
| 40 | Battery circuit |
| 5 | Inverter |
| 50 | Inverter circuit |
| T0, T1 | Transistors of the inverter |
| 6 | Oscillating circuit |
| 60 | Coil |
| 61 | Coil circuit |
| 62 | Susceptor circuit |
| 63, 64 | Voltage sensors |
| C1, C2 | Capacitors of the oscillating circuit |
| fₒₚ | Operating frequency |
| fᵢ | Initial resonant frequency |
| fᵣ | Resonant frequency |
| 7 | Susceptor |
| T_{d} | Determined temperature of the susceptor |
| Tₜ | Target temperature of the susceptor |
| 8 | Boost converter |
| 80 | Boost converter circuit |
| 81 | Inductor of the boost converter |
| 82 | Capacitor of the boost converter |
| 83 | Current sensor |
| 84 | Voltage sensor |
| 9 | Controller |
| T2 | Active switch of the boost converter |
| T3 | Passive switch of the boost converter |
| Vᵢₙ | Input voltage of the boost converter |
| Vₒᵤₜ | Output voltage of the boost converter |
| Sᵢₙ | Initialization step |
| Sₚ | Power delivery mode |
| S_{Ti} | Temperature identification mode |

1-21. (canceled)

22. A method for controlling heating of a susceptor of an aerosol-generating device, the susceptor being inductively heated by an oscillating circuit driven by an inverter, a boost converter being connected between a power supply unit and said inverter, the boost converter being configured to step-up voltage from an input voltage supplied from the power supply unit to an output voltage delivered to the inverter, wherein said method comprises a power delivery mode of the aerosol-generating device and a temperature identification mode of the aerosol-generating device in which an amount of power supplied to the inverter is lower than an amount of power supplied during the power delivery mode, the method further comprising a step of determining a temperature of the susceptor, and setting the output voltage delivered from the boost converter to the inverter during the power delivery mode depending on the determined temperature of the susceptor.

23. The method according to claim 22, wherein the step of determining the temperature of the susceptor is based on a determined resonant frequency of the oscillating circuit.

24. The method according to claim 22, wherein the inverter comprises two transistors, said step of determining the temperature of the susceptor comprising the following sub-steps:
operating only one of the two transistors of the inverter;
determining a resonant frequency of the oscillating circuit during the temperature identification mode; and
determining the temperature of the susceptor based on said determined resonant frequency.

25. The method according to claim 22, wherein the step of determining the temperature of the susceptor is based on a determined maximum value of an indicative electrical value in the oscillating circuit.

26. The method according to claim 25, wherein the indicative electrical value is a voltage across a capacitor of the oscillating circuit.

27. The method according to claim 25, wherein said step of determining the temperature of the susceptor comprises the following sub-steps:
determining a maximum value of the indicative electrical value in the oscillating circuit for a range of frequencies during the temperature identification mode;
determining a global maximum value from determined maximum values; and
determining the temperature of the susceptor based on said global maximum value.

28. The method according to claim 25, wherein the inverter comprises two transistors, said step of determining the temperature of the susceptor further comprising the sub-step of operating both transistors during the temperature identification mode at a reduced duty cycle.

29. The method according to claim 22, wherein the power delivery mode and the temperature identification mode are alternated during operating of the aerosol-generating device.

30. The method according to claim 22, wherein the temperature identification mode is run at regular intervals of time.

31. The method according to claim 22, further comprising a comparison step performed before the setting step, in which the determined temperature of the susceptor is compared with a target temperature, the output voltage being set at a value depending on the determined temperature and the target temperature.

32. The method according to claim 31, wherein the aerosol-generating device comprises a controller configured to control the output voltage of the boost converter in order to bring the temperature of the susceptor to the target temperature, said controller being tuned to be overdamped, and wherein the output voltage is set at a maximum predefined voltage when the determined temperature of the susceptor is inferior or equal to a threshold value.

33. The method according to claim 32, wherein the threshold value ranges between 60% and 85% of the target temperature.

34. The method according to claim 31, wherein the aerosol-generating device comprises a controller configured to control the output voltage of the boost converter in order to bring the temperature of the susceptor to the target temperature, said controller being tuned to be underdamped, and wherein the output voltage is set so that the temperature of the susceptor overshoots the target temperature at a beginning of the heating of the susceptor.

35. The method according to claim 32, wherein the controller is a PID controller, a model-based controller and/or a model-predictive controller.

36. The method according to claim 22, wherein the output voltage is set substantially equal to or less than a predetermined voltage when the determined temperature of the susceptor is equal to the target temperature.

37. The method according to claim 22, wherein the boost converter is an asynchronous boost converter.

38. The method according to claim 22, wherein the boost converter is a synchronous boost converter.

39. The method according to claim 22, wherein the boost converter comprises an active switch, said active switch being a MOSFET transistor.

40. The method according to claim 22, wherein the boost converter comprises a passive switch, said passive switch being a MOSFET transistor.

41. The method according to claim 22, wherein the boost converter is configured to step-up voltage from an input voltage ranging from 3 to 4.2V to a desired output voltage e.g. at least equal to 8V.

42. An aerosol-generating device comprising:
a power supply unit;
an induction heatable susceptor;
an oscillating circuit arranged to generate a time varying electromagnetic field for inductively heating the susceptor;
an inverter configured to drive the oscillating circuit-;
a boost converter connected on one side to the power supply unit and on another side to the inverter; and
a controller adapted to implement the method for controlling the heating of the susceptor according to claim 22.
